(19) United States
(12) Patent Application Publication
(10) Pub. No.: US 2021/0177740 A1
(43) Pub. Date: Jun. 17, 2021

(54) TRANSPORE DELIVERY OF CANNABINOID AND USES THEREOF

(71) Applicant: Joel STUDIN, Roslyn, NY (US)

(72) Inventor: Joel STUDIN, Roslyn, NY (US)

(21) Appl. No.: 16/711,280

(22) Filed: Dec. 11, 2019

(51) Int. Cl. A61K 9/00; A61K 31/05; A61K 36/73

(52) U.S. Cl. CPC A61K 9/0014; A61K 36/73; A61K 31/05

(57) ABSTRACT

The invention relates to novel cannabinoid pharmaceutical compositions and methods for transpore delivery of cannabinoid to a subject for treating a disease or for recreational use.

### FIELD OF THE INVENTION

The invention relates to novel cannabinoid pharmaceutical compositions and methods for transpore delivery of cannabinoid to a subject for treating diseases or for recreational use. More specifically to a topical cannabinoid composition and its use for systemic and/or topical transpore delivery of cannabinoid to a subject for treating diseases or for recreational use.

### BACKGROUND

Cannabinoids (“CBD”) are a class of diverse chemical compounds that act on cannabinoid receptors, which are part of the endocannabinoid system found in cells that alter neurotransmitter release in the brain. Medical uses of CBD include the treatment of nausea due to chemotherapy, spasticity, and possibly neuropathic pain. However, delivery of CBD can be difficult.

Transdermal drug delivery can be attempted through various dosage forms, for example, patches, creams and ointments. Each dosage form has its respective limitations. Patches are difficult to apply on curved surfaces, cumbersome, and uncomfortable. They also cause pain when peeled off and have poor aesthetic appeal. Dermal patches also exhibit reliability problems, not sticking predictably in different climates and degrees of skin oiliness. This limits the efficacy of transdermal drug delivery via patches. It is well-established that amplification of transdermal bioavailability by occlusion alone is inadequate to treat many diseases.

Semisolid preparations, like creams and ointments, overcome some of these drawbacks but have other limitations. Creams and ointments do not allow persistent contact with the skin, can be easily wiped off, and need to be applied repeatedly. They also leave a sticky and greasy feel after application, which may lead to poor patient compliance. Moreover, when creams and ointments are applied to skin, there is a risk of the drug transferring to another person.

Additionally, as with transdermal patches, it is well-established that bioavailability of the active drug is often inadequate for treatment when delivered by creams and ointments. In these situations, only injections have been effective. Injections, however, are painful, expensive, and poorly tolerated by patients, especially when there is a need for repeat injections overtime.

While transdermal delivery methods and formulations continue to be developed to address public health associated with a variety of diseases, there is a need for an improved method of transpore drug delivery of drugs, bypassing the skin surface without an injection, which permits painless, comfortable and invisible application and thereby improve drug delivery and patient compliance.

### BRIEF SUMMARY OF THE INVENTION

According to various aspects and aspects, the present disclosure is directed to a novel cannabinoid pharmaceutical composition for transpore delivery and methods of use.

In one aspect, the liquid cannabinoid composition is formulated to be delivered through skin pores, bypassing the stratum corneum of the skin, and into the systemic circulation of a subject.

In one aspect, the liquid cannabinoid composition is formulated to be delivered through skin pores, bypassing the stratum corneum of the skin, and into the skin of a subject without substantially passing into the systemic circulation of a subject.

The present disclosure provides a liquid pharmaceutical composition comprising about 0.5% to about 20% by weight of a cannabinoid, the composition, when administered to a subject, achieves one or more of the following: a) forms a solid or semi-solid film; and b) has a thickness of about 0.1 μm to about 10 μm in solid or semi-solid form. In one aspect, the composition further achieves the following: c) provides a mean Tₘₐₓ of the cannabinoid from about 0.5 hour to about 10 hours. In one aspect, the composition further achieves the following: c) delivers into the systemic circulation of the subject at least 10% of the total amount of cannabinoid in the composition. In one aspect, the composition further achieves the following: c) topically delivers into the skin of the subject at least 50% of the total amount of cannabinoid in the composition.

In one aspect, the composition is administered to a subject for treating a disease. In one aspect, the disease is pain.

In one aspect, the liquid cannabinoid composition further comprises a polymer and an organic solvent. In one aspect, the liquid composition comprises pyroxilin, ether, and alcohol.

In one aspect, after the liquid cannabinoid composition is applied to the skin, the film has a thickness of about 1 μm to about 5 μm in solid form.

In one aspect, the liquid cannabinoid composition seeps into skin pores in liquid form and creates a biomechanical integration with the inside surface of said skin pores in solid form.

In one aspect, the liquid cannabinoid composition further comprises hemp oil.

In one aspect, the cannabinoid is selected from the group consisting of cannabinol, cannabinolic acid, Δ(9)-tetrahydrocannabinol, Δ(9)-tetrahydrocannabinolic acid, cannabidiol, Δ(9)-tetrahydrocannabidiolic acid, Δ(8)-tetrahydrocannabinol, Δ(8)-tetrahydrocannabinolic acid, Δ(8)-tetrahydrocannabidiol, Δ(8), tetrahydrocannabidiolic acid, Δ(9)-tetrahydrocannabivarin, cannabigerol, cannabidigerolic acid, cannabichromene, cannabichromenic acid, cannabicyclol, cannabicyclolic acid, cannabielsoin, cannabitriol, and nabilone, or combinations thereof. In one aspect, the cannabinoid is cannabidiol.

In one aspect, the cannabinoid liquid composition is administered to treat cutaneous pain, joint pain, visceral pain, back pain, or neuropathic pain.

The present disclosure also provides a method for systemic transpore delivery of a cannabinoid to a subject. In one aspect, the method for systemic transpore delivery of a cannabinoid to a subject is for treating a disease. In one aspect, the disease can be any condition treatable by a cannabinoid. In one aspect, the disease is pain.

In one aspect, the method for systemic transport delivery comprises applying a liquid composition comprising about 0.5% to about 20% by weight of a cannabinoid to the skin of the subject, wherein the composition, when applied to the subject, achieves one or more of the following: a) forms a solid or semi-solid film; b) has a thickness of about 0.1 μm to about 10 μm in solid or semi-solid form; c) provides a mean Tₘₐₓ of cannabinoid from about 0.5 hour to about 20 hours; and d) provides a maximum serum cannabinoid concentration (Cₘₐₓ) of about 1.0 ng/mL to about 150.0 ng/mL; wherein said composition seeps into skin pores in liquid form and creates a biomechanical integration with the inside surface of said skin pores in solid form.

In one aspect, the film formed in the method has a thickness of about 1 μm to about 5 μm.

In one aspect, the area of skin application is about 1 cm² to about 500 cm².

In one aspect, when applied to the skin of the subject, the cannabinoid composition provides a maximum serum cannabinoid concentration (Cₘₐₓ) following administration of about 20 ng/mL to about 100 ng/mL.

In one aspect, the cannabinoid used in the method is selected from cannabinol, cannabinolic acid, Δ(9)-tetrahydrocannabinol, Δ(9)-tetrahydrocannabinolic acid, cannabidiol, Δ(9)-tetrahydrocannabidiolic acid, Δ(8)-tetrahydrocannabinol, Δ(8)-tetrahydrocannabinolic acid, Δ(8)-tetrahydrocannabidiol, Δ(8), tetrahydrocannabidiolic acid, Δ(9)-tetrahydrocannabivarin, cannabigerol, cannabidigerolic acid, cannabichromene, cannabichromenic acid, cannabicyclol, cannabicyclolic acid, cannabielsoin, cannabitriol, and nabilone, or combinations thereof.

In one aspect, the pain treated by the method disclosed herein is joint pain, visceral pain, or neuropathic pain.

The present disclosure also provides a method for topical transpore delivery of a cannabinoid to a subject's skin. In one aspect, the method for systemic transpore delivery of a cannabinoid to a subject is for treating a disease. In one aspect, the disease can be any condition treatable by a cannabinoid. In one aspect, the disease is pain.

The method comprises applying a liquid composition comprising about 0.5% to about 20% by weight of a cannabinoid to the skin of the subject, wherein the composition, when applied to the subject, achieves one or more of the following: a) forms a solid or semi-solid film; b) has a thickness of about 0.1 μm to about 10 μm in solid or semi-solid form; and c) provides a maximum serum cannabinoid concentration (Cₘₐₓ) of less than 1.0 ng/mL; wherein said composition seeps into skin pores in liquid form and creates a biomechanical integration with the inside surface of said skin pores in solid form.

In one aspect, the film formed in the method has a thickness of about 1 μm to about 5 μm.

In one aspect, the area of skin application is about 1 cm² to about 500 cm².

In one aspect, the cannabinoid used in the method is selected from cannabinol, cannabinolic acid, Δ(9)-tetrahydrocannabinol, Δ(9)-tetrahydrocannabinolic acid, cannabidiol, Δ(9)-tetrahydrocannabidiolic acid, Δ(8)-tetrahydrocannabinol, Δ(8)-tetrahydrocannabinolic acid, Δ(8)-tetrahydrocannabidiol, Δ(8), tetrahydrocannabidiolic acid, Δ(9)-tetrahydrocannabivarin, cannabigerol, cannabidigerolic acid, cannabichromene, cannabichromenic acid, cannabicyclol, cannabicyclolic acid, cannabielsoin, cannabitriol, and nabilone, or combinations thereof. In one aspect, the cannabinoid is cannabinol.

In one aspect, the pain treated by the method disclosed herein is cutaneous pain.

In one aspect, the present disclosure also provides a method for systemic transport delivery of a cannabinoid for recreational use comprising applying a liquid composition comprising about 0.5% to about 20% by weight of a cannabinoid to the skin of the subject, wherein the composition, when applied to the subject, achieves one or more of the following: a) forms a solid or semi-solid film; b) has a thickness of about 0.1 μm to about 10 μm in solid or semi-solid form; c) provides a mean Tₘₐₓ of cannabinoid from about 0.5 hour to about 20 hours; and d) provides a maximum serum cannabinoid concentration (Cₘₐₓ) of about 1.0 ng/mL to about 150.0 ng/mL; wherein said composition seeps into skin pores in liquid form and creates a biomechanical integration with the inside surface of said skin pores in solid form.

### BRIEF DESCRIPTION OF THE DRAWINGS

FIG. 1A provides a sectional view of the structure of skin pores.

FIG. 1B provides a sectional view of the transpore delivery of liquid cannabinoid compositions into the skin pores.

### DETAILED DESCRIPTION OF THE INVENTION

### Definition

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

### Definition of General Terms

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

The term “substantial” as used herein is used in its ordinary sense, including, without limitation, an amount greater than 50 percent.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

### Definition of Specific Terms

The term “pharmaceutically acceptable” as used herein generally refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “excipient” refers to any substance, not itself a therapeutic agent, which may be used in a composition for delivery of an active therapeutic agent to a subject or combined with an active therapeutic agent (e.g., to create a pharmaceutical composition) to improve its handling or storage properties or to permit or facilitate formation of a dose unit of the composition (e.g., formation of a hydrogel which may then be optionally incorporated into a patch). Excipients include, by way of illustration and not limitation, binders, disintegrants, taste enhancers, solvents, thickening or gelling agents (and any neutralizing agents, if necessary), penetration enhancers, solubilizing agents, wetting agents, antioxidants, lubricants, emollients, substances added to mask or counteract a disagreeable odor, fragrances or taste, substances added to improve appearance or texture of the composition and substances used to form hydrogels. Any such excipients can be used in any dosage forms according to the present disclosure. The foregoing classes of excipients are not meant to be exhaustive but merely illustrative as a person of ordinary skill in the art would recognize that additional types and combinations of excipients could be used to achieve the desired goals for delivery of a drug.

The term “systemic transpore delivery” generally refers to delivery of pharmaceutical or therapeutic composition in and/or through the skin pores into the systemic circulation of a subject.

The term “topical transpore delivery” generally refers to delivery of a pharmaceutical or therapeutic composition in and/or through the skin pores to a skin tissue (e.g., epidermis or dermis) of the body.

The term “recreational use” as used herein generally refers to a drug used without medical justification for its psychoactive effects often in the belief that occasional use of such a substance is not habit-forming or addictive.

The terms “skin tissue” and “skin” are used interchangeably in the present disclosure and refer to the superficial layer of skin including the epidermis and dermis and all dermal structures such as sensory nerve endings, blood vessels, sweat glands, etc.

The term “Cₘₐₓ” as used herein generally refers to the maximum plasma concentration of a drug after it is administered to a subject.

The term “Cₘᵢₙ” as used herein generally refers to the lowest concentration reached by a drug before the next dose is administered.

The term “Tₘₐₓ” as used herein generally refers to the time required to reach the maximal plasma concentration (“Cₘₐₓ”) after administration of a drug.

The term “C_{avg}” as used herein generally refers to the mean plasma concentration of a drug achieved by transpore delivery.

The term “treat,” “treating,” or “treatment” as used herein generally refers to the administration of a composition to a subject for therapeutic purposes of eliminating, reducing, relieving, reversing, and/or ameliorating a disease or condition and/or symptoms associated therewith. Although not precluded, treating a disease or condition does not require that the disease, condition, or symptoms associated therewith be completely eliminated, including the treatment of acute or chronic signs, symptoms and/or malfunctions. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition, “treatment” therefore also includes relapse prophylaxis or phase prophylaxis.

The term “administration” or “administering” as used herein refers to the act of a physician or other medical professional prescribing a pharmaceutical composition of the disclosure herein for a subject.

The term “mean” generally refers to an average value in a patient population. For example, a “mean Cₘₐₓ” refers to an average of the maximum plasma concentrations of a drug in a patient population.

The term “occlusive film” generally refers to a solid or semi-solid film that is an impermeable thin layer of material that covers the skin.

The term “serum concentration” generally refers to the amount of a drug or other compound in the circulation, both bound to proteins and unbound, the latter of which generally corresponds to the therapeutically active fraction.

The term “bioavailability” generally refers to the rate and extent to which the active ingredient is absorbed from a drug product and becomes available at the site of action.

“Bioequivalence” is a term in pharmacokinetics generally used to assess the expected in vivo biological equivalence of two proprietary preparations of a drug. Two pharmaceutical products are bioequivalent if they are pharmaceutically equivalent and their bioavailabilities (rate and extent of availability) after administration in the same molar dose are similar to such a degree that their effects, with respect to both efficacy and safety, can be expected to be essentially the same.

### Cannabinoid Pharmaceutical Compositions

The present disclosure provides a liquid composition which comprises at least one film forming polymer. In one aspect, the liquid composition comprises at least two film-forming polymers. Examples of film-forming polymers include, but are not limited to, cellulose nitrates, cellulose esters, cellulose ethers, cellulose esters-ethers, cellulose acylate, polyquaternium hyaluronic acid, or any combinations thereof. In one aspect, the film-forming polymer is pyroxylin.

In certain aspects, the total weight percentage of the one or more film forming polymers in the composition is from about 1% to about 10%, from about 3% to about 10%, from about 5% to about 10%, from about 7% to about 10%, from about 1% to about 8%, from about 3% to about 8%, from about 5% to about 8%, from about 7% to about 8%, from about 1% to about 6%, from about 3% to about 6%, from about 5% to about 6%, from about 1% to about 4%, from about 2% to about 4%, or from about 1% to about 2%. In one aspect, the total weight percentage of the one or more film forming polymers is about 1%, about 2%, about 3%, about 4%, about 5%, about 6%, about 7%, about 8%, about 9%, or about 10%.

In one aspect, the liquid cannabinoid composition further comprises nitrocellulose, ether and alcohol. In certain aspects, the total weight percentage of nitrocellulose, ether and alcohol is from about 50% to about 99%, from about 60% to about 99%, from about 70% to about 99%, from about 80% to about 99% from about 90% to about 99% from about 50% to about 90%, from about 60% to about 90%, from about 70% to about 90%, from about 80% to about 90%, from about 50% to about 80%, from about 60% to about 80%, from about 70% to about 80%, from about 50% to about 70%, or from about 60% to about 70%.

In one aspect, the liquid cannabinoid composition comprises pyroxylin, ether and alcohol. In certain aspects, the total weight percentage of pyroxylin, ether and alcohol is from about 50% to about 99%, from about 60% to about 99%, from about 70% to about 99%, from about 80% to about 99%, from about 90% to about 99%, from about 50% to about 90%, from about 60% to about 90%, from about 70% to about 90%, from about 80% to about 90%, from about 50% to about 80%, from about 60% to about 80%, from about 70% to about 80%, from about 50% to about 70%, or from about 60% to about 70%.

In one aspect, the liquid cannabinoid composition comprises about 1% to about 10% of pyroxylin by weight. In one aspect, the composition comprises about 1%, about 2%, about 3%, about 4%, about 5%, about 6%, about 7%, about 8%, about 9%, about 10% of pyroxylin by weight. In one aspect, the composition comprises about 40% to about 75% of ether by weight. In one aspect, the composition comprises about 40% to about 50%, about 40% to about 60%, about 50% to about 60%, about 50% to about 75%, or about 60% to about 75% of ether by weight. In one aspect, the composition comprises about 20% to about 30% of alcohol. In one aspect, the composition comprises about 20%, about 21%, about 22%, about 23%, about 24%, about 25%, about 26%, about 27%, about 28%, about 29%, or about 30% of alcohol by weight. Examples of ethers include, but are not limited to, diethyl ether and polyoxyetheylene lauryl ether. Examples of alcohol include, but are not limited to, ethanol and isopropanol. In one aspect, the proportion of the weight of alcohol to the weight of ether is about 1:4, about 1:3.5, about 1:3, about 1:2.5, or about 1:2. In one aspect, the liquid composition comprises 4 g nitrocellulose in 100 mL of a mixture of 25 mL alcohol and 75 mL ether.

In one aspect, the liquid cannabinoid composition has a sufficient amount of ether and alcohol to dissolve all the ingredients and is a clear solution. In one aspect, the ratio of ether and alcohol is from about 0.001 to about 1,000, about 0.01 to about 500, about 0.1 to about 100, about 0.1 to about 50, about 0.2 to about 40, about 0.3 to about 30, about 0.4 to about 20, about 0.5 to about 15, about 0.6 to about 10, about 0.7 to about 5, about 0.8 to about 3, about 0.9 to about 2, or about 1 to about 1.5.

In one aspect, the weight percentage of pyroxylin in the liquid cannabinoid composition is from about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 9%, about 1% to about 8%, about 1% to about 7%, about 1% to about 6%, about 1% to about 5%, about 1% to about 4%, about 1% to about 3%, or about 1% to about 2%.

In yet other aspects, the liquid cannabinoid composition comprises one or more plasticizers. In certain aspects, the total weight percentage of the one or more plasticizers is from about 1% to about 20%, from about 5% to about 20%, from about 10% to about 20%, from about 15% to about 20%, from about 1% to about 16%, from about 5% to about 16%, from about 10% to about 16%, from about 1% to about 12%, from about 5% to about 12%, from about 8% to about 12%, from about 1% to about 8%, or from about 4% to about 8%. In one aspect, the total weight percentage of the plasticizer is about 1%, about 2%, about 3%, about 4%, about %, about 6%, about 7%, about 8%, about 9%, about 10%, about 11%, about 12%, about 13%, about 14%, about 15%, about 16%, about 17%, about 18%, about 19%, or about 20%.

Examples of suitable plasticizers for the liquid drug compositions include, but are not limited to, polyethylene glycol, propylene glycol, polyesters (e.g. poly (lactic acid) and poly(lactide-co-glycolide)), polyesteramides, diesters/triesters of acids, diesters/triesters of alcohols, and combinations thereof.

In one aspect, the liquid cannabinoid composition comprises a penetration enhancer. Penetration enhancers can interact with the lipid domain of the stratum corneum, disrupting these, and causing fluidization. Examples of penetration enhancers include, but are not limited to, dimethylsulphoxide, azone, pyrrolidones, fatty acids, fatty alcohols, peptides and trypsin. The total weight percentage of the penetration enhancers in the liquid composition is from about 0 to about 20%. In certain aspects, the weight percentage of the penetration enhancer in the liquid composition is from about 0 to about 18%, from about 0 to about 16%, from about 0 to about 14%, from about 0 to about 12%, from about 0 to about 10%, from about 0 to about 8%, from about 0 to about 6%, from about 0 to about 4%, from about 0 to about 2%, from about 0 to about 1%. In one aspect, the liquid composition does not include a penetration enhancer.

In one aspect, the liquid cannabinoid composition comprises a surfactant. Examples of surfactants include, but are not limited to, alkylglucosides, alkylmaltosides, alkylthioglucosides, lauryl macrogolglycerides, fatty acids, lower alcohol fatty acid esters, polyoxyethylene alkylphenols, polyethylene glycol fatty acids esters, polypropylene glycol fatty acid esters, glycerol fatty acid esters, acetylated, glycerol fatty acid esters, polyethylene glycol glycerol fatty acid esters, polyglyceryl fatty acid esters, polyoxyethylene sorbitan fatty acid esters, polyoxyethylene glycerides, polyoxyethylene sterols, polyoxyethylene vegetable oils, polyoxyethylene hydrogenated vegetable oils, reaction mixtures of polyols and at least one member of the group consisting of fatty acids, vegetable oils, hydrogenated vegetable oils, and sterols, sugar esters, sugar ethers, sucroglycerides, fatty acid salts, bile salts, phospholipids, phosphoric acid esters, carboxylates, sulfates, sulfonates, or a combination thereof.

In one aspect, the liquid cannabinoid composition comprises at least one pharmaceutically acceptable excipient. Suitable excipients that may be used in the liquid compositions discussed herein are known in the art and include, but are not limited to, polypeptides, synthetic polymers, liposomes, transfersomes, ethosomes, niosomes, solid lipid nanoparticles, penetration enhancers, solubilizers such as C₂ to C₈ straight and branched chain alcohols, diols and triols, moisturizers and humectants such as glycerine, amino acids and amino acid derivatives, polyaminoacids and derivatives, pyrrolidone carboxylic acids and its salts and derivatives, surfactants such as sodium laureth sulfate, sorbitan monolaurate, emulsifiers such as cetyl alcohol, stearyl alcohol, thickeners such as methyl cellulose, ethyl cellulose, hydroxymethylcellulose, hydroxypropylcellulose, polyvinylpyrollidone, polyvinyl alcohol and acrylic polymers, or combinations of any of the above.

In one aspect, the liquid cannabinoid composition comprises about 0.1% to about 20% by weight of a pharmaceutically acceptable excipient. In one aspect, the liquid cannabinoid composition comprises about 1% to about 20%, about 3% to about 20%, about 5% to about 20%, about 8% to about 20%, about 10% to about 20%, about 12% to about 20%, about 15% to about 20%, about 18% to about 20%, about 0.1% to about 15%, about 1% to about 15%, about 3% to about 15%, about 5% to about 15%, about 8% to about 15%, about 10% to about 15%, about 12% to about 15%, about 0.1% to about 12%, about 1% to about 12%, about 3% to about 12%, about 5% to about 12%, about 8% to about 12%, about 10% to about 12%, about 8% to about 10%, about 0.1% to about 8%, about 1% to about 8%, about 3% to about 8%, about 5% to about 8%, about 0.1% to about 3%, about 1% to about 3%, or about 0.1% to about 1% by weight of a pharmaceutically acceptable excipient.

In one aspect, the liquid cannabinoid composition comprises at least one organic solvent. In one aspect, the liquid composition comprises at least two organic solvents. In one aspect, the liquid composition comprises two volatile solvents. In one aspect, the volatile solvent is, but not limited to, alkane, alkene, ether, ester, alcohol, nitrile, or acetone. In one aspect, a suitable organic solvent includes, but is not limited to, ethyl acetate, ether, ethyl alcohol, isopropyl alcohol, propylene glycol, hexane, heptane, toluene, and combinations thereof. In one aspect, the liquid composition comprises ethyl alcohol and ether.

In one aspect, the thickness of the film after application ranges from about 0.1 μm to about 10 μm, from about 0.1 μm to about 5 μm, from about 0.1 μm to about 2 μm, from about 0.5 μm to about 10 μm, from about 0.5 μm to about 5 μm, from about 0.5 μm to about 2 μm, from about 1 μm to about 10 μm, from about 1 μm to about 5 μm, from about 1 μm to about 2 μm, about 3 μm to about 10 μm, from about 3 μm to about 5 μm, about 5 μm to about 10 μm, from about 7 μm to about 10 μm. In one aspect, the thickness of the film ranges from about 3 μm to about 4 μm.

In one aspect, the film formed by the liquid cannabinoid composition is an occlusive film. Occlusion refers to an impermeable film. An occlusive film blocks diffusional water loss from the skin, thereby increasing hydration of the stratum corneum. Maintenance of the structural integrity of the stratum corneum is critical to the skin's barrier function. Increasing stratum corneum hydration reduces the skin's barrier efficiency. Therefore, an occlusive film enhances the penetration of a topically administered drug through skin pores.

In one aspect, the liquid cannabinoid composition comprises about 0.001% to about 50%, about 0.01% to about 40%, about 0.1% to about 30%, about 0.1% to about 25%, about 0.1% to about 20%, about 0.5% to about 20%, about 0.5% to about 15%, about 0.5% to about 10%, about 1% to about 10%, about 3% to about 10%, about 5% to about 10%, about 7% to about 10%, about 9% to about 10%, about 0.001% to about 8%, about 0.01% to about 8%, about 0.1% to about 8%, about 0.5% to about 8%, about 1% to about 8, about 3% to about 8%, about 5% to about 8%, about 7% to about 8, about 0.001% to about 6, about 0.01% to about 6%, about 0.1% to about 6%, about 0.5% to about 6%, about 1% to about 6%, about 3% to about 6%, about 5% to about 6%, about 0.001% to about 4%, about 0.01% to about 4%, about 0.1% to about 4%, about 0.5% to about 4%, about 1% to about 4%, about 3% to about 4%, about 0.001% to about 2%, about 0.01% to about 2%, about 0.1% to about 2%, about 0.5% to about 2%, about 1% to about 2%, about 0.001% to about 1%, about 0.01% to about 1%, about 0.1% to about 1%, or about 0.5% to about 1% by weight of cannabinoid. In one aspect, the liquid cannabinoid composition comprises about 0.001%, about 0.01%, about 0.1%, about 0.5%, about 1%, about 5%, about 10%, about 15%, about 20%, about 25%, about 30%, about 35%, about 40%, about 45%, or about 50% by weight of cannabinoid.

In one aspect, the liquid cannabinoid composition further comprises hemp oil. Hemp oil generally includes cannabidiol in varying amounts depending upon how the hemp oil is prepared. As used herein, “hemp oil” generally refers to oil formed by leaching or extracting substantially nonaqueous compounds from the hemp plant. The term “hemp oil” may generally refer to any of these oils, “extracts,” “essences,” “essential oils,” and may refer to any type of hemp oil, including “hash oil” and other oils that may include a variety of compounds found in hemp plants, also known as marijuana plants. Hemp and marijuana are well known as being a source for tetrahydrocannabinol and other mind altering cannabinoids.

In one aspect, one or more cannabinoids in the hemp oil can be enriched. In one aspect, the liquid cannabinoid composition comprises hemp oil which is enriched with one or more of following cannabinoids: cannabiripsol, cannabitriol, cannabidiol, tetrahydrocannabinol, cannabinol, cannabigerol, cannabichromene, cannabielsoin, and cannabicitran. In one aspect, the hemp oil is enriched with cannabidiol.

In one aspect, the liquid cannabinoid composition comprises about 0.001% to about 50%, about 0.01% to about 40%, about 0.1% to about 30%, about 0.1% to about 25%, about 0.1% to about 20%, about 0.5% to about 20%, about 0.5% to about 15%, about 0.5% to about 10%, about 1% to about 10%, about 3% to about 10%, about 5% to about 10%, about 7% to about 10%, about 9% to about 10%, about 0.001% to about 8%, about 0.01% to about 8%, about 0.1% to about 8%, about 0.5% to about 8%, about 1% to about 8%, about 3% to about 8%, about 5% to about 8%, about 7% to about 8%, about 0.001% to about 6%, about 0.01% to about 6%, about 0.1% to about 6%, about 0.5% to about 6%, about 1% to about 6%, about 3% to about 6%, about 5% to about 6%, about 0.001% to about 4%, about 0.01% to about 4%, about 0.1% to about 4%, about 0.5% to about 4%, about 1% to about 4%, about 3% to about 4%, about 0.001% to about 2%, about 0.01% to about 2%, about 0.1% to about 2%, about 0.5% to about 2%, about 1% to about 2%, about 0.001% to about 1%, about 0.01% to about 1%, about 0.1% to about 1%, or about 0.5% to about 1% by weight of cannabinoid. In one aspect, the liquid cannabinoid composition comprises about 0.001%, about 0.01%, about 0.1%, about 0.5%, about 1%, about 5%, about 10%, about 15%, about 20%, about 25%, about 30%, about 35%, about 40%, about 45%, or about 50% by weight of hemp oil.

In one aspect, the cannabinoid in the disclosure herein can encompass a chemical compound that activates any mammalian cannabinoid receptor, for example human CB₁ receptor or human CB₂ receptor. As used herein a chemical compound that activates a mammalian cannabinoid receptor includes agonists of said receptor. The skilled person can readily determine whether a compound is a cannabinoid receptor agonist or activator using assays known in the art, for example using a suitable [³⁵S]GTPγS binding assay (see, for example Griffin et al, Journal of Pharmacology and Experimental Therapeutics, 285(2), pp. 553-560, 1998). The cannabinoids can be naturally occurring (such as, for example, endocannabinoids or phytocannabinoids) or they can be synthetic. Based on their structure similarity, cannabinoids can be divided into 11 subclasses: cannabigerol (CBG-type), (−)-Δ(9)-tetrahydrocannabinol (Δ(9)-THC-type), cannabidiol (CBD-type), cannabichromene (CBC-type), cannabinol (CBN-type), (−)-Δ(8)-tetrahydrocannabinol (Δ(8)-THC-type), cannabicyclol (CBL-type), cannabidiol (CBND-type), cannabielsoin (CBE-type), cannabitriol (CBT-type) and miscellaneous type. See Citti, C., et. al., (2019) Cannabinoid Profiling of Hemp Seed Oil by Liquid Chromatography Coupled to High-Resolution Mass Spectrometry, Frontiers in Plant Science 10: 120. About 120 cannabinoids have been identified. See Hanuš L. O., et. al., (2016) Phytocannabinoids: a unified critical inventory. Nat. Prod. Rep. 33, 1357-1392. The cannabinoids disclosed therein are incorporated by reference therein.

In one aspect, the cannabinoid in the disclosure herein is selected from the cannabinoid classes consisting of cannabigerol, (−)-Δ(9)-tetrahydrocannabinol, cannabidiol, cannabichromene, cannabinol, (−)-Δ(8)-tetrahydrocannabinol, cannabicyclol, cannabidiol, cannabielsoin, cannabitriol, miscellaneous type, and combinations thereof.

In one aspect, the cannabinoid in the disclosure herein is selected from the group consisting of cannabinol, cannabinolic acid, Δ(9)-tetrahydrocannabinol, Δ(9)-tetrahydrocannabinolic acid, cannabidiol, Δ(9)-tetrahydrocannabidiolic acid, Δ(8)-tetrahydrocannabinol, Δ(8)-tetrahydrocannabinolic acid, Δ(8)-tetrahydrocannabidiol, Δ(8), tetrahydrocannabidiolic acid, Δ(9)-tetrahydrocannabivarin, cannabigerol, cannabidigerolic acid, cannabichromene, cannabichromenic acid, cannabicyclol, cannabicyclolic acid, cannabielsoin, cannabitriol, and nabilone, or combinations thereof.

In one aspect, the liquid cannabinoid composition is formulated to be delivered through skin pores, bypassing the stratum corneum of the skin, and into the systemic circulation of a subject.

In one aspect, the liquid cannabinoid composition is formulated to be delivered through skin pores to a skin tissue of a subject. In one aspect, the liquid cannabinoid composition is formulated to be delivered through skin pores, bypassing the stratum corneum of the skin, and into the skin of a subject. In one aspect, the liquid cannabinoid composition is formulated to be delivered through skin pores, bypassing the stratum corneum of the skin, and into the skin of a subject without substantially passing into the systemic circulation of a subject. In one aspect, the liquid cannabinoid composition is formulated to deliver greater than 50% of cannabinoid through skin pores, bypassing the stratum corneum of the skin, and into the skin of a subject.

In one aspect, the liquid cannabinoid composition is formulated such that, when administered to a subject, it achieves one or more of the following: a) forms a solid or semi-solid film; and b) has a thickness of about 0.1 μm to about 10 μm in solid or semi-solid form. In one aspect, the liquid cannabinoid composition is administered to a subject for treating a disease. In one aspect, the disease is pain.

In one aspect, the liquid cannabinoid composition is formulated such that, when administered to a subject, it achieves one or more of the following: a) forms a solid or semi-solid film; b) has a thickness of about 0.1 μm to about 10 μm in solid or semi-solid form; and c) provides a mean Tₘₐₓ of the cannabinoid from about 0.5 hour to about 10 hours. In one aspect, the mean Tₘₐₓ of the cannabinoid ranges from 0.5 hour to about 9 hours, from 0.5 hour to about 8 hours, from 0.5 hour to about 7 hours, from 0.5 hour to about 6 hours, from 1 hour to about 6 hours, from 2 hours to about 6 hours, from 3 hours to about 6 hours, from 4 hours to about 6 hours, from 5 hours to about 6 hours, or from 5.5 hours to about 6 hours.

In one aspect, the liquid cannabinoid composition is formulated such that, when administered to a subject, it delivers the cannabinoid into the systemic circulation of the subject at least 0.1%, at least 0.5%, at least 1%, at least 5%, at least 10%, at least 15%, at least 20%, at least 25%, at least 30%, at least 35%, at least 40%, at least 45%, or at least 50%, of total amount of cannabinoid in the composition. In one aspect, the liquid cannabinoid composition is formulated such that, when administered to a subject, it delivers the cannabinoid into the systemic circulation of the subject up to 10%, up to 9%, up to 8%, up to 7%, up to 6%, up to 5%, up to 4%, up to 3%, up to 2%, up to 1%, up to 0.5%, or up to 0.1% of total amount of cannabinoid in the composition.

In one aspect, the liquid cannabinoid composition is formulated such that, when administered to a subject, it provides a maximum serum cannabinoid concentration (Cₘₐₓ) of about 1.0 ng/mL to about 150.0 ng/mL, about 5.0 ng/mL to about 150.0 ng/mL, about 10.0 ng/mL to about 150.0 ng/mL, about 15.0 ng/mL to about 150.0 ng/mL, about 20.0 ng/mL to about 150.0 ng/mL, about 25.0 ng/mL to about 150.0 ng/mL, about 30.0 ng/mL to about 150.0 ng/mL, about 35.0 ng/mL to about 150.0 ng/mL, about 40.0 ng/mL to about 150.0 ng/mL, about 45.0 ng/mL to about 150.0 ng/mL, about 50.0 ng/mL to about 150.0 ng/mL, about 55.0 ng/mL to about 150.0 ng/mL, about 60.0 ng/mL to about 150.0 ng/mL, about 65.0 ng/mL to about 150.0 ng/mL, about 70.0 ng/mL to about 150.0 ng/mL, about 75.0 ng/mL to about 150.0 ng/mL, about 80.0 ng/mL to about 150.0 ng/mL, about 85.0 ng/mL to about 150.0 ng/mL, about 90.0 ng/mL to about 150.0 ng/mL, about 95.0 ng/mL to about 150.0 ng/mL, about 100.0 ng/mL to about 150.0 ng/mL, about 110.0 ng/mL to about 150.0 ng/mL, about 120.0 ng/mL to about 150.0 ng/mL, about 130.0 ng/mL to about 150.0 ng/mL, about 140.0 ng/mL to about 150.0 ng/mL, 20.0 ng/mL to about 140.0 ng/mL, 20.0 ng/mL to about 130.0 ng/mL, 20.0 ng/mL to about 120.0 ng/mL, 20.0 ng/mL to about 110.0 ng/mL, 20.0 ng/mL to about 100.0 ng/mL, 20.0 ng/mL to about 90.0 ng/mL, 20.0 ng/mL to about 80.0 ng/mL, 20.0 ng/mL to about 70.0 ng/mL, 20.0 ng/mL to about 60.0 ng/mL, 20.0 ng/mL to about 50.0 ng/mL, 20.0 ng/mL to about 40.0 ng/mL, 20.0 ng/mL to about 30.0 ng/mL, or 20.0 ng/mL to about 25.0 ng/mL.

In one aspect, the liquid cannabinoid composition is formulated such that, when administered to a subject, it delivers to the skin of a subject at least 0.1%, at least 0.5%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of total amount of cannabinoid in the composition. In one aspect, the liquid cannabinoid composition is formulated such that, when administered to a subject, it delivers into the systemic circulation of the subject less than 50%, less than 45%, less than 40%, less than 35%, less than 30%, less than 25%, less than 20%, less than 15%, less than 10%, less than 5%, or less than 1% total amount of cannabinoid in the composition.

In one aspect, the liquid cannabinoid composition is formulated such that, when administered to a subject, it provides a maximum serum cannabinoid concentration (Cₘₐₓ) less than 1.0 ng/mL, less than 0.9 ng/mL, less than 0.8 ng/mL, less than 0.7 ng/mL, less than 0.6 ng/mL, less than 0.5 ng/mL, less than 0.4 ng/mL, less than 0.3 ng/mL, less than 0.2 ng/mL, or less than 0.1 ng/mL.

In one aspect, the liquid cannabinoid composition of the present disclosure can be used to treat pains. In one aspect, the pain is cutaneous pain, joint pain, visceral pain, back pain, or neuropathic pain.

### Method of Transpore Delivery

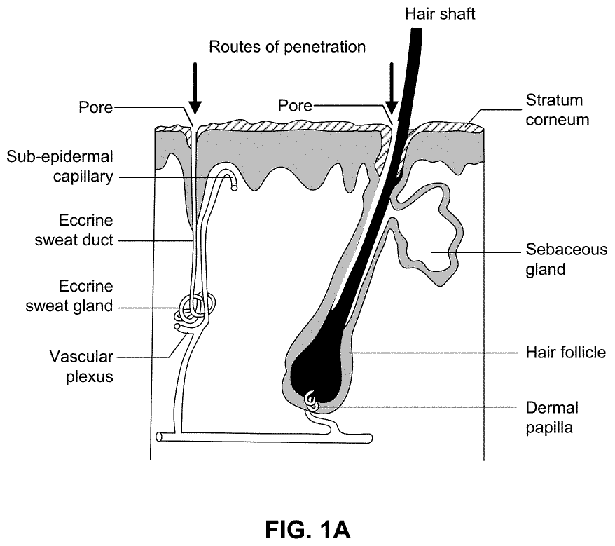

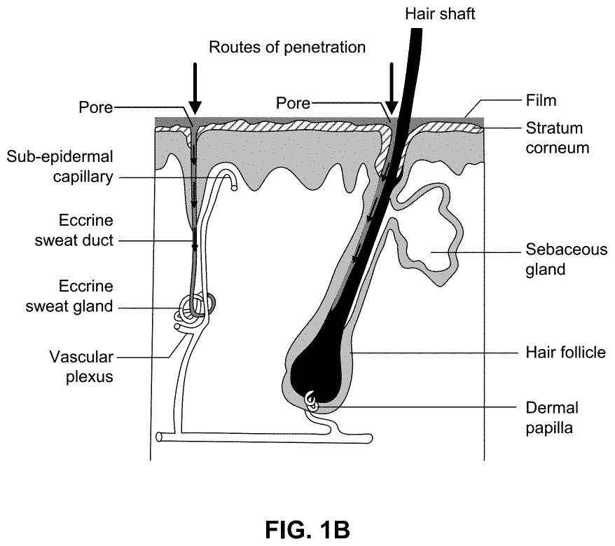

The skin is an important route for the delivery of drugs. Drug delivery through skin manages to avoid the variable absorption and metabolic breakdown associated with oral treatments and injections as the compound enters the systemic circulation without passing through the liver. Human skin is comprised of four main layers: the stratum corneum (SC); the viable epidermis; the overlying dermis; and the innermost subcutaneous tissues (hypodermis). FIG. 1 presents an illustration of normal human skin. The external layer of the skin (SC) functions as an effective barrier, and is essential for protection of the internal milieu from the external environment. Permeation through the SC is the rate limiting step in the dermal or transdermal delivery of drugs.

One route of transdermal delivery involves the movement of compounds into the skin through skin pores. Physiologically speaking, skin pores comprise the tiny ostia from either pilosebaceous follicles or eccrine sweat glands. A pilosebaceous follicle is a unit consisting of a hair follicle and a sebum-producing sebaceous gland. Eccrine sweat glands secrete water to the skin, where it cools the body by evaporation. There are two types of skin pores with different sizes: a pilosebaceous follicle has a diameter of approximately 40-80 μm, and an eccrine sweat gland has a diameter of approximately 5-10 μm. Hydrophilic and high molecular weight molecules, as well as particle-based drug delivery systems, can penetrate the skin through skin pores.

The present disclosure describes a method of transpore delivery of a cannabinoid via skin pores. The method comprises applying a liquid composition that dries out to a solid or semi-solid film to the skin of a subject. When applied to the skin, the liquid composition seeps into the skin pores as well as covers the surface. The liquid dries as the solvent in the composition evaporates. The remaining polymer materials in the composition absorb local moisture, swell, and dry to a solid or semi-solid film. Thus, the composition creates a biomechanical integration with the microstructure of the skin. The film is tangible, yet barely visible, avoiding compliance issues and adheres in a peg-lock manner with the skin pores.

In one aspect, the liquid cannabinoid composition once dried, can also be described as an intrapore drug-eluting stent or stent-like structure.

In one aspect, the subject is preferably a mammal such as a non-primate, e.g., cow, pig, horse, cat, dog, rat, and a primate, e.g., a monkey such as a Cynomolgous monkey and a human.

Skin pores originate in the dermal tissue but are accessible on the surface of the skin. In essence, skin pores provide a passage way for an active agent to directly reach the dermis without having to traverse the intact barrier of the SC. In transpore delivery, the pharmaceutically active ingredient in the liquid composition travels through the skin pores to arrive at the viable epidermis and the dermis. The film that dries out from the liquid composition is sufficiently thin to contour the shape of each skin pore. This allows the film to sufficiently contact with the skin pores and enhances the efficiency of transpore delivery.

In one aspect, upon application to skin, the liquid dries rapidly to form a clear, long-lasting, highly durable elastomeric film, adhering to the contours of the skin and providing a uniform film. Once brushed on the skin, the volatile components, diethyl ether and ethyl alcohol rapidly evaporate, leaving a thin transparent film on the skin. As the film adheres to the skin and dries, the cannabinoid impregnated film permeates the pores of the skin, creating a transpore delivery system for the cannabinoid.

The effect of transpore delivery of the cannabinoid can be tested in variety of methods, for example, collecting pharmaceutical kinetic data, such as Cₘₐₓ, Cₘᵢₙ, Tₘₐₓ, Tₘᵢₙ, or clearance, of cannabinoid in vivo after application of the liquid composition of present disclosure on the skin of a subject. In one aspect, the effect of transpore delivery of the cannabinoid can also be determined by vasodilation in peripheral vasculature. See Graham J. D., et al (1973). Cardiovascular and Respiratory Effects of Cannabis in Cat and Rat, Br. J. Pharmacol, 49, 1-10 (revealing a drop in systemic blood pressure and pulse as well as respiratory rate is dose dependent on cannabis-extract); also see Cavero I., Ertel R., et. al., (1972) Effects of (−)-9-trans-tetrahydrocannabinol on regional blood flow in anesthetized dogs, Eur. J. Pharmacol. 20, 373-376 (revealing a relationship between reduction of cardiac output and administration of Δ-9-THC).

In one aspect, the liquid cannabinoid composition is applied to the skin by any common applicator such as a brush, roll, squeeze tube, sprayer or eye drop type of apparatus used to apply compositions to the skin. The compositions may also be applied by impregnating a porous base with the composition and wiping the resultant composition onto the skin area or where the porous base includes an adhesive, securing the porous base to the skin adjacent to the skin area, wherein the liquid composition is placed on the area to be treated.

In one aspect, the liquid cannabinoid composition used in the method of the present disclosure is a relatively low or high viscosity liquid which can be applied directly and accurately onto the skin area and does not require the application of additional pressure or rubbing as do certain creams and ointments that have been previously utilized. The term “viscosity” is the measure of fluid friction. A highly viscous material is one that possesses a great deal of internal friction, and will not pour or spread as easily as material of lesser viscosity. A typical range of suitable viscosities for the present liquid composition would be, for example, 0.1 to 5000 mPas, preferably 1 to 1000 mPas at 20° C.

In one aspect, the liquid cannabinoid composition is applied to a skin area including, but not limited to, one or more of an axilla, shoulder, arm, neck, abdomen, buttock, chest, back, or thigh. In one aspect, the liquid cannabinoid composition is applied a skin membrane such as the cornea, oral, sublingual, or buccal mucosa.

In one aspect, the area of skin to which the composition is applied is from about 1 cm² to about 1000 cm², from about 1 cm² to about 500 cm², from about 1 cm² to about 300 cm², from about 1 cm² to about 200 cm², from about 1 cm² to about 100 cm², from about 1 cm² to about 50 cm², from about 1 cm² to about 25 cm², from about 1 cm² to about 10 cm², or from about 1 cm² to about 5 cm². In one aspect, the area of skin is from about 1 cm² to about 500 cm².

One of the advantages of the film-forming composition is that once the liquid composition dries to an occlusive film, the film can remain on the skin for days to achieve a prolonged release of the active ingredient. Unlike traditional drug-release patches that are thick and not visually appealing, the film is so thin that it is barely noticeable as well as does not interfere with most daily activities of the patient. Because the film can stay on the skin for a prolonged time, it also eliminates the cumbersome need for repeated application by the patient.

In some aspects, the occlusive film formed by the liquid cannabinoid composition is kept on the skin for from 1 to 7 days, from 1 to 5 days, from 1 to 3 days, from 3 to 7 days, from 3 to 5 days, or from 5 to 7 days. The composition can be reapplied as needed if the film peels off the skin area. In one aspect, the occlusive film is kept on the skin for 2-7 days.

In one aspect, the amount of the liquid cannabinoid composition that is brushed on to the skin in a single dose is from about 0.05 to about 10 ml, from about 0.5 to about 5 ml, from about 0.5 to about 3 ml, from about 0.5 to about 1 ml, from about 0.5 to about 0.5 ml, from about 0.5 to about 10 ml, from about 0.5 to about 5 ml, from about 0.5 to about 3 ml, from about 0.5 to about 1 ml, from about 0.5 to about 1 ml, from about 1 to about 10 ml, from about 1 to about 5 ml, from about 1 to about 3 ml, from about 3 to about 10 ml, from about 3 to about 5 ml, from about 5 to about 10 ml, from about 5 to about 8 ml, or from about 7 to about 10 ml. In certain aspects, the amount of liquid composition that is applied to the skin is a daily dose of about 0.05 ml, about 0.1 ml, about 0.5 ml, about 1 ml, about 2 ml, about 3 ml, about 4 ml, about 5 ml, about 6 ml, about 7 ml, about 8 ml, about 9 ml, or about 10 ml. In one aspect, the amount of the composition that is brushed on to the skin is from about 0.5 to about 5 ml.

In one aspect, the amount of the cannabinoid that is applied to the skin is a single dose from about 0.1 mg to about 10 mg, from about 0.1 mg to about 5 mg, from about 0.1 mg to about 3 mg, from about 0.1 mg to about 1 mg, from about 0.1 mg to about 0.5 mg, from 0.5 mg to about 10 mg, from about 0.5 to about 5 mg, from about 0.5 mg to about 3 mg, from about 0.5 mg to about 1 mg, from about 1 mg to about 10 mg, from about 1 mg to about 5 mg, from about 1 mg to about 3 mg, from about 3 mg to about 10 mg, from about 3 mg to about 7 mg, from about 3 mg to about 5 mg, from about 5 mg to about 10 mg, from about 5 mg to about 7 mg, from about 7 mg to about 10 mg, from about 0.05 mg to about 15 mg, from about 0.05 mg to about 10 mg, from about 0.05 mg to about 5 mg, from about 0.05 mg to about 1 mg, from about 0.05 mg to about 0.5 mg, from about 0.1 mg to about 20 mg, from about 0.1 mg to about 15 mg, from about 0.5 mg to about 20 mg, from about 0.5 mg to about 15 mg, from about 1 mg to about 20 mg, from about 1 mg to about 15 mg, from about 3 mg to about 20 mg, from about 3 mg to about 15 mg, from about 5 mg to about 20 mg, from about 5 mg to about 15 mg, from about 7 mg to about 20 mg, from about 7 mg to about 15 mg, from about 10 mg to about 20 mg, from about 10 mg to about 15 mg, from about 15 mg to about 20 mg, from about 5 mg to about 1000 mg, from about 5 mg to about 500 mg, from about 5 mg to about 100 mg, from about 5 mg to about 50 mg, from about 10 mg to about 1000 mg, from about 10 mg to about 500 mg, from about 10 mg to about 100 mg, from about 10 mg to about 50 mg, from about 50 to about 1000 mg, from about 50 to about 500 mg, from about 50 mg to about 100 mg, from about 100 mg to about 1000 mg, from about 100 mg to about 500 mg, or from about 500 mg to about 1000 mg.

In one aspect, the liquid cannabinoid composition, when administered to a subject, results in about 1% to about 99% of the cannabinoid entering the systemic circulation of the patient after about 8 to about 10 hours of contact on the skin. In one aspect, the composition results in about 1% to about 80%, about 2% to about 70%, about 5% to about 60%, about 10% to about 50%, about 11% to about 45%, about 12% to about 40%, about 13% to about 35%, about 14% to about 30%, about 15% to about 25%, about 15% to about 22%, about 15% to about 20%, about 15% to about 18%, or about 15% to about 16% of the active ingredient entering the systemic circulation of the patient after 8 hours of contact on the skin. In one aspect, the liquid composition results in about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, about 95%, or about 99% of cannabinoid entering the systemic circulation of the patient after 8 hours of contact on the skin.

In one aspect, the method disclosed herein delivers the liquid cannabinoid composition through skin pores, bypassing the stratum corneum of the skin, and into the systemic circulation of a subject.

In one aspect, the present disclosure provides a method for systemic transpore delivery of a cannabinoid to a subject for treating a disease. In one aspect, the disease can be any condition treatable by a cannabinoid. In one aspect, the disease is pain. In one aspect, the pain is joint pain, visceral pain, back pain, or neuropathic pain. The method comprises applying a liquid composition comprising about 0.5% to about 20% by weight of a cannabinoid to the skin of the subject, wherein the composition, when applied to the subject, achieves one or more of the following: a) forms a solid or semi-solid film; b) has a thickness of about 0.1 μm to about 10 μm in solid or semi-solid form; c) provides a mean Tₘₐₓ of cannabinoid from about 0.5 hour to about 20 hours; and d) provides a maximum serum cannabinoid concentration (Cₘₐₓ) of about 1.0 ng/mL to about 150.0 ng/mL; wherein said composition seeps into skin pores in liquid form and creates a biomechanical integration with the inside surface of said skin pores in solid form.

In one aspect, the present disclosure also provides a method for systemic transport delivery of a cannabinoid for recreational use comprising applying a liquid composition comprising about 0.5% to about 20% by weight of a cannabinoid to the skin of the subject, wherein the composition, when applied to the subject, achieves one or more of the following: a) forms a solid or semi-solid film; b) has a thickness of about 0.1 μm to about 10 μm in solid or semi-solid form; c) provides a mean Tₘₐₓ of cannabinoid from about 0.5 hour to about 20 hours; and d) provides a maximum serum cannabinoid concentration (Cₘₐₓ) of about 1.0 ng/mL to about 150.0 ng/mL; wherein said composition seeps into skin pores in liquid form and creates a biomechanical integration with the inside surface of said skin pores in solid form.

In one aspect, the area of skin application is about 1 cm² to about 500 cm².

In one aspect, the film has a thickness of about 1 μm to about 5 μm.

In one aspect, the method disclosed herein provides a mean Tₘₐₓ of cannabinoid from about 0.5 hour to about 20 hours, about 1 hour to about 20 hours, about 2 hours to about 20 hours, about 3 hours to about 20 hours, about 4 hours to about 15 hours, about 4 hours to about 12 hours, about 4 hours to about 10 hours, about 4 hours to about 8 hours, or about 4 hours to about 6 hours.

In one aspect, the method disclosed herein provides a maximum serum cannabinoid concentration (Cₘₐₓ) of about 1.0 ng/mL to about 150.0 ng/mL, about 5.0 ng/mL to about 150.0 ng/mL, about 10.0 ng/mL to about 150.0 ng/mL, about 15.0 ng/mL to about 150.0 ng/mL, about 20.0 ng/mL to about 150.0 ng/mL, about 25.0 ng/mL to about 150.0 ng/mL, about 30.0 ng/mL to about 150.0 ng/mL, about 35.0 ng/mL to about 150.0 ng/mL, about 40.0 ng/mL to about 150.0 ng/mL, about 45.0 ng/mL to about 150.0 ng/mL, about 50.0 ng/mL to about 150.0 ng/mL, about 55.0 ng/mL to about 150.0 ng/mL, about 60.0 ng/mL to about 150.0 ng/mL, about 65.0 ng/mL to about 150.0 ng/mL, about 70.0 ng/mL to about 150.0 ng/mL, about 75.0 ng/mL to about 150.0 ng/mL, about 80.0 ng/mL to about 150.0 ng/mL, about 85.0 ng/mL to about 150.0 ng/mL, about 90.0 ng/mL to about 150.0 ng/mL, about 95.0 ng/mL to about 150.0 ng/mL, about 100.0 ng/mL to about 150.0 ng/mL, about 110.0 ng/mL to about 150.0 ng/mL, about 120.0 ng/mL to about 150.0 ng/mL, about 130.0 ng/mL to about 150.0 ng/mL, about 140.0 ng/mL to about 150.0 ng/mL, 20.0 ng/mL to about 140.0 ng/mL, 20.0 ng/mL to about 130.0 ng/mL, 20.0 ng/mL to about 120.0 ng/mL, 20.0 ng/mL to about 110.0 ng/mL, 20.0 ng/mL to about 100.0 ng/mL, 20.0 ng/mL to about 90.0 ng/mL, 20.0 ng/mL to about 80.0 ng/mL, 20.0 ng/mL to about 70.0 ng/mL, 20.0 ng/mL to about 60.0 ng/mL, 20.0 ng/mL to about 50.0 ng/mL, 20.0 ng/mL to about 40.0 ng/mL, 20.0 ng/mL to about 30.0 ng/mL, or 20.0 ng/mL to about 25.0 ng/mL.

In one aspect, the method disclosed herein provides a steady state ratio of serum cannabinoid Cₘₐₓ to Cₘᵢₙ of about 10.0 or less based on single subject administration. In one aspect, the steady state ratio of serum testosterone Cₘₐₓ to Cₘᵢₙ is about 1 to about 9, about 1 to about 8, about 1 to about 7, about 1 to about 6, about 1 to about 5, about 1 to about 4, about 1 to about 3, or about 1 to about 2.

In one aspect, the method disclosed herein can be used to treat any condition associated with pain, including, but not limited to joint pain, visceral pain, or neuropathic pain. In one aspect, the method disclosed herein can be used to treat pain in patients with psoriatic arthritis, fibromyalgia, trigeminal neuralgia, scleroderma, shingles and related pain generating conditions.

In one aspect, the present disclosure provides a method for topical transpore delivery of a cannabinoid to a subject's skin for treating a disease. In one aspect, the disease can be any condition treatable by a cannabinoid. In one aspect, the disease is pain.

In one aspect, the method for topical transpore delivery comprises applying a liquid composition comprising about 0.5% to about 20% by weight of a cannabinoid to the skin of the subject, wherein the composition, when applied to the subject, achieves one or more of the following: a) forms a solid or semi-solid film; b) has a thickness of about 0.1 μm to about 10 μm in solid or semi-solid form; and c) provides a maximum serum cannabinoid concentration (Cₘₐₓ) of less than 1.0 ng/mL; wherein said composition seeps into skin pores in liquid form and creates a biomechanical integration with the inside surface of said skin pores in solid form.

In one aspect, the method disclosed herein delivers the liquid cannabinoid composition through skin pores, bypassing the stratum corneum of the skin, and into the skin of a subject. In one aspect, the method disclosed herein delivers the liquid cannabinoid composition through skin pores, bypassing the stratum corneum of the skin, and into the skin of a subject. In one aspect, the method disclosed herein delivers the liquid cannabinoid composition through skin pores, bypassing the stratum corneum of the skin, and into the systemic circulation of the subject less than 50%, less than 45%, less than 40%, less than 35%, less than 30%, less than 25%, less than 20%, less than 15%, less than 10%, less than 5%, less than 1%, less than 0.9%, less than 0.8%, less than 0.7%, less than 0.6%, less than 0.5%, less than 0.4%, less than 0.3%, less than 0.2%, or less than 0.1% of total amount of cannabinoid in the composition. In one aspect, the method disclosed herein delivers the liquid cannabinoid composition through skin pores, bypassing the stratum corneum of the skin, and into the systemic circulation of the subject up to 1%, up to 0.9%, up to 0.8%, up to 0.7%, up to 0.6%, up to 0.5%, up to 0.4%, up to 0.3%, up to 0.2%, or up to 0.1% of total amount of cannabinoid in the composition.

In one aspect, the area of skin application is about 1 cm² to about 500 cm².

In one aspect, the film has a thickness of about 1 μm to about 5 μm.

In one aspect, the method disclosed herein provides a maximum serum cannabinoid concentration (Cₘₐₓ) less than 1.0 ng/mL, less than 0.9 ng/mL, less than 0.8 ng/mL, less than 0.7 ng/mL, less than 0.6 ng/mL, less than 0.5 ng/mL, less than 0.4 ng/mL, less than 0.3 ng/mL, less than 0.2 ng/mL, or less than 0.1 ng/mL.

In one aspect, the method disclosed herein can be used to treat a variety of skin disorders that involve pain, such as postherpetic neuralgia, diabetic neuropathy, psoriasis, cluster headache, postmastectomy pain syndrome, rhinopathy, oral mucositis, cutaneous allergy, detrusor hyperreflexia, loin pain/hematuria syndrome, neck pain, amputation stump pain, reflex sympathetic dystrophy, pain due to skin tumor and arthritis, including rheumatoid arthritis, osteoarthritis, diabetic neuropathy, psoriasis, pruritus (itching), cluster headache, post-surgical pain, tendonitis, oral pain, and pain caused by injury, amongst others. The method can be used to treat aches and pains of muscles and joints. In one aspect, the method disclosed herein can be used to treat cutaneous pain.

### Examples

The following examples are provided to promote a clearer understanding of certain aspects of the present invention, and are in no way meant as a limitation thereon. From the above discussion and the Examples, one skilled in the art can ascertain the essential characteristics of the invention, and without departing from the spirit and scope thereof, can make various changes and modifications to adapt the invention to various uses and conditions. As a result, the invention is not limited by the illustrative examples set forth hereinbelow, but rather is defined by the claims appended hereto.

General procedure for transpore delivery of drugs: a cannabinoid is dissolved in a nitrocellulose (collodion) solution. Upon application to skin, the liquid dries rapidly to form a clear, long-lasting, highly durable elastomeric film, adhering to the contours of the skin and providing a uniform film. Once brushed on the skin, the volatile components, such as diethyl ether and ethyl alcohol, rapidly evaporate, leaving a thin transparent film on the skin. As the film adheres to the skin and dries, the drug-impregnated film permeates the pores of the skin, creating a transpore delivery system for the cannabinoid.

### Examples 1-4: Liquid Cannabidiol Compositions

Table 1 provides formulations of four liquid cannabidiol compositions.

TABLE 1

| Ingredients | Example 1 | Example 2 | Example 3 | Example 4 |
|---|---|---|---|---|
| Nitrocellulose | 10 wt. % | 15 wt. % | 15 wt. % | 20 wt. % |
| Cannabidiol | 10 wt. % | 10 wt. % | 15 wt. % | 20 wt. % |
| Ethyl Alcohol | 60 wt. % | 55 wt. % | 50 wt. % | 45 wt. % |
| Diethyl Ether | 20 wt. % | 20 wt. % | 20 wt. % | 15 wt. % |
| Total mL | 10 | 10 | 10 | 10 |

### Examples 5-8: Liquid Δ(9)-Tetrahydrocannabinol Compositions

Table 2 provides formulations of four liquid Δ(9)-tetrahydrocannabinol compositions.

TABLE 2

| Ingredients | Example 5 | Example 6 | Example 7 | Example 8 |
|---|---|---|---|---|
| Nitrocellulose | 10 wt. % | 15 wt. % | 15 wt. % | 25 wt. % |
| Δ(9)-Tetrahydro- | 15 wt. % | 15 wt. % | 20 wt. % | 20 wt. % |
| cannabinol | | | | |
| Ethyl Alcohol | 60 wt. % | 55 wt. % | 45 wt. % | 40 wt. % |
| Diethyl Ether | 15 wt. % | 15 wt. % | 20 wt. % | 15 wt. % |
| Total mL | 10 | 10 | 10 | 10 |

### Examples 9-12: Liquid Cannabinol Compositions

Table 3 provides formulations of four liquid cannabinol compositions.

TABLE 3

| Ingredients | Example 9 | Example 10 | Example 11 | Example 12 |
|---|---|---|---|---|
| Nitrocellulose | 10 wt. % | 15 wt. % | 15 wt. % | 25 wt. % |
| Cannabinol | 5 wt. % | 10 wt. % | 15 wt. % | 20 wt. % |
| Ethyl Alcohol | 70 wt. % | 60 wt. % | 45 wt. % | 40 wt. % |
| Diethyl Ether | 15 wt. % | 15 wt. % | 25 wt. % | 15 wt. % |
| Total mL | 10 | 10 | 10 | 10 |

### Examples 13-16: Liquid Cannabitriol Compositions

Table 4 provides formulations of four liquid cannabitriol compositions.

TABLE 4

| Ingredients | Example 13 | Example 14 | Example 15 | Example 16 |
|---|---|---|---|---|
| Nitrocellulose | 10 wt. % | 15 wt. % | 15 wt. % | 25 wt. % |
| Cannabitriol | 2 wt. % | 5 wt. % | 10 wt. % | 15 wt. % |
| Ethyl Alcohol | 73 wt. % | 70 wt. % | 60 wt. % | 45 wt. % |
| Diethyl Ether | 15 wt. % | 10 wt. % | 15 wt. % | 15 wt. % |
| Total mL | 10 | 10 | 10 | 10 |

### Example 17

The formulation of Example 4 is used in a pharmacokinetic study. 10 mL of liquid composition is brushed onto the neck of a healthy volunteer. After 24 hours, the solid film formed on the neck of the volunteer is washed off. Plasma concentrations of cannabidiol are analyzed using LC/MS/MS.

Peak plasma concentration (Cmax), time to peak plasma concentration (tmax) and area under the curve from t=0 to t=24 h (AUC) are calculated. The results will be expected in Table 5.

TABLE 5
Pharmacokinetic Parameters

| | Formulation 4 |
|---|---|
| Cₘₐₓ (ng/mL) | 110 |
| Tₘₐₓ (h) | 8 |
| AUC (ng/mL h) | 125 |

What is claimed is:

1. A liquid pharmaceutical composition comprising about 0.5% to about 20% by weight of a cannabinoid,
the composition, when administered to a subject, achieves one or more of the following:
a) forms a solid or semi-solid film; and
b) has a thickness of about 0.1 μm to about 10 μm in solid or semi-solid form.

2. The composition of claim 1, further comprising a polymer and an organic solvent.

3. The composition of claim 1, wherein the composition further achieves the following:
c) delivers into the systemic circulation of the subject up to 10% of the total amount of cannabinoid in the composition.

4. The composition of claim 1, wherein said composition seeps into skin pores in liquid form and creates a biomechanical integration with the inside surface of said skin pores in solid form.

5. The composition of claim 2, further comprising hemp oil.

6. The composition of claim 1, wherein the cannabinoid is selected from the group consisting of cannabinol, cannabinolic acid, Δ(9)-tetrahydrocannabinol, Δ(9)-tetrahydrocannabinolic acid, cannabidiol, Δ(9)-tetrahydrocannabidiolic acid, Δ(8)-tetrahydrocannabinol, Δ(8)-tetrahydrocannabinolic acid, Δ(8)-tetrahydrocannabidiol, Δ(8), tetrahydrocannabidiolic acid, Δ(9)-tetrahydrocannabivarin, cannabigerol, cannabidigerolic acid, cannabichromene, cannabichromenic acid, cannabicyclol, cannabicyclolic acid, cannabielsoin, cannabitriol, and nabilone, or combinations thereof.

7. The composition of claim 1, wherein the composition further achieves the following:
c) topically delivers into the skin of the subject at least 50% of the total amount of cannabinoid in the composition.

8. The composition of claim 1, wherein the composition is administered for treating a disease, wherein the disease is pain.

9. A method for systemic transpore delivery of a cannabinoid to a subject comprising
applying a liquid composition comprising about 0.5% to about 20% by weight of a cannabinoid to the skin of the subject,
wherein the composition, when applied to the subject, achieves one or more of the following:
a) forms a solid or semi-solid film;
b) has a thickness of about 0.1 μm to about 10 μm in solid or semi-solid form;
c) provides a mean Tₘₐₓ of cannabinoid from about 0.5 hour to about 20 hours; and
d) provides a maximum serum cannabinoid concentration (Cₘₐₓ) of about 1.0 ng/mL to about 150.0 ng/mL;
wherein said composition seeps into skin pores in liquid form and creates a biomechanical integration with the inside surface of said skin pores in solid form.

10. The method of claim 9, wherein the film has a thickness of about 1 μm to about 5 μm.

11. The method of claim 9, wherein said area of skin application is about 1 cm² to about 500 cm².

12. The method of claim 9, wherein said composition, when administered to the subject, provides a maximum serum cannabinoid concentration (Cₘₐₓ) following administration of about 20 ng/mL to about 100 ng/mL.

13. The method of claim 12, wherein the cannabinoid is selected from the group consisting of cannabinol, cannabinolic acid, Δ(9)-tetrahydrocannabinol, Δ(9)-tetrahydrocannabinolic acid, cannabidiol, Δ(9)-tetrahydrocannabidiolic acid, Δ(8)-tetrahydrocannabinol, Δ(8)-tetrahydrocannabinolic acid, Δ(8)-tetrahydrocannabidiol, Δ(8), tetrahydrocannabidiolic acid, Δ(9)-tetrahydrocannabivarin, cannabigerol, cannabidigerolic acid, cannabichromene, cannabichromenic acid, cannabicyclol, cannabicyclolic acid, cannabielsoin, cannabitriol, and nabilone, or combinations thereof.

14. The method of claim 9, wherein the composition is administered for treating a disease, wherein the disease is pain.

15. A method for topical transpore delivery of a cannabinoid to a subject's skin comprising
applying a liquid composition comprising about 0.5% to about 20% by weight of a cannabinoid to the skin of the subject,
wherein the composition, when applied to the subject, achieves one or more of the following:
a) forms a solid or semi-solid film;
b) has a thickness of about 0.1 μm to about 10 μm in solid or semi-solid form; and
c) provides a maximum serum cannabinoid concentration (Cₘₐₓ) of less than 1.0 ng/mL;
wherein said composition seeps into skin pores in liquid form and creates a biomechanical integration with the inside surface of said skin pores in solid form.

16. The method of claim 15, wherein the film has a thickness of about 1 μm to about 5 μm.

17. The method of claim 15, wherein said area of skin application is about 1 cm² to about 500 cm².

18. The method of claim 15, wherein the cannabinoid is selected from the group consisting of cannabinol, cannabinolic acid, Δ(9)-tetrahydrocannabinol, Δ(9)-tetrahydrocannabinolic acid, cannabidiol, Δ(9)-tetrahydrocannabidiolic acid, Δ(8)-tetrahydrocannabinol, Δ(8)-tetrahydrocannabinolic acid, Δ(8)-tetrahydrocannabidiol, Δ(8), tetrahydrocannabidiolic acid, Δ(9)-tetrahydrocannabivarin, cannabigerol, cannabidigerolic acid, cannabichromene, cannabichromenic acid, cannabicyclol, cannabicyclolic acid, cannabielsoin, cannabitriol, and nabilone, or combinations thereof.

19. The method of claim 18, wherein the composition is administered for treating a disease, wherein the disease is pain.

20. The method of claim 18, wherein the cannabinoid is cannabinol.
